(19) United States
(12) Patent Application Publication
(10) Pub. No.: US 2026/0283994 A1
(43) Pub. Date: Sep. 24, 2026

(54) ORAL ADMINISTRATION COMPOSITION, SOFT CAPSULE FORMULATION AND METHOD FOR PRODUCING ORAL ADMINISTRATION COMPOSITION

(71) Applicant: SUNTORY HOLDINGS LIMITED, Osaka-shi, Osaka (JP)

(72) Inventors: Fumiko Oikawa, Soraku-gun (JP); Yohei Kikuchi, Soraku-gun (JP); Yu Ipponmatsu, Soraku-gun (JP)

(73) Assignee: SUNTORY HOLDINGS LIMITED, Osaka-shi, Osaka (JP)

(21) Appl. No.: 19/471,897

(22) PCT Filed: Apr. 1, 2024
(86) PCT No.: PCT/JP2024/013480

§ 371 Date: Oct. 2, 2025

(30) Foreign Application Priority Data
Apr. 5, 2023 (JP) 2023-061430

(51) Int. Cl. A61K 31/202; A61K 9/48; A61K 31/047; A61K 36/28; A61K 47/14

(52) U.S. Cl. CPC A61K 31/202; A61K 9/4891; A61K 31/047; A61K 36/28; A61K 47/14

(57) ABSTRACT

The present invention aims to provide an oral composition that contains an arachidonic acid-class compound and at least one compound selected from the group consisting of lutein, zeaxanthin, and fatty acid esters thereof and that is less prone to component separation. The present invention relates to an oral composition containing: a component (A): an arachidonic acid-class compound; a component (B): at least one compound selected from the group consisting of lutein, zeaxanthin, and fatty acid esters thereof, and a component (C): a glycerol fatty acid ester (s), wherein the glycerol fatty acid ester (s) is a monoglyceride and/or a diglyceride containing a C12-C24 saturated fatty acid as a constituent fatty acid.

### TECHNICAL FIELD

The present invention relates to an oral composition and a method for producing the same. The present invention also relates to a soft capsule containing the oral composition.

### BACKGROUND ART

Carotenoids are anti-oxidative substances that are produced, for example, by plants, algae, and microorganisms and that are rich in vegetables and fruits, and are known for their strong anti-oxidative effect (Non-Patent Literature 1 and Non-Patent Literature 2). According to a report, the human brain contains 16 types of carotenoids (Non-Patent Literature 3). In particular, lutein and zeaxanthin, which are types of carotenoids, rich in vegetables such as Komatsuna (Non-Patent Literature 4) are known for their neuroprotective effect owing to the anti-oxidative effect in the cranial nervous system (Non-Patent Literature 5 and Non-Patent Literature 6).

Arachidonic acid (hereinafter also referred to as “ARA”), which is contained in meat, fish and shellfish, eggs, and the like, is also known to have many physiological activities. Regarding compositions containing ARA, for example, Patent Literature 1 discloses a composition containing a compound that contains ARA as a constituent fatty acid, wherein the composition has an action that reduces a decline in higher brain functions caused by organic brain damage.

### CITATION LIST

### Patent Literature

Patent Literature 1: JP 2016-172770 A

### Non-Patent Literature

Non-Patent Literature 1: Manochkumar, J. et al. Phytomedicine 91, 153676 (2021)
Non-Patent Literature 2: Maoka, T. J. Nat. Med. 74, 1-16 (2020)
Non-Patent Literature 3: Craft, N. E. et al. J. Nutr. Health Aging 8, 156-162 (2004)
Non-Patent Literature 4: Aizawa, K. & Inakuma, T. Food Sci. Technol. Res. 13, 247-252 (2007)
Non-Patent Literature 5: Singhrang, N. et al. J. Biomed. Pharmacother. 103, 582-587 (2018)
Non-Patent Literature 6: Hu, Y. et al. J. Nutr. Sci. Vitaminol. (Tokyo). 67, 234-242 (2021)

### SUMMARY OF INVENTION

### Technical Problem

Arachidonic acid and carotenoids have various physiological activities, and are therefore in high demand as health foods. However, it has been found that compounding arachidonic acid and at least one compound selected from the group consisting of lutein, zeaxanthin, and fatty acid esters thereof causes a failure in maintaining the stability of a composition and is likely to cause separation of the components. This separation of the components of the composition has posed a problem with the undesirable appearance of products using the composition and a problem with the difficulty in accurately compounding a desired amount of an active ingredient in products using the composition, for example.

The present invention aims to provide an oral composition that contains an arachidonic acid-class compound and at least one compound selected from the group consisting of lutein, zeaxanthin, and fatty acid esters thereof and that is less prone to component separation.

### Solution to Problem

As a result of extensive studies to solve the above problems, the present inventors have found that use of a monoglyceride and/or a diglyceride containing a C12-C24 saturated fatty acid as a constituent fatty acid leads to oral composition that contains an arachidonic acid-class compound and at least one compound selected from the group consisting of lutein, zeaxanthin, and fatty acid esters thereof and that is less prone to separation.

The present invention encompasses the following oral compositions, for example.
[1] An oral composition containing: a component (A): an arachidonic acid-class compound; a component (B): at least one compound selected from the group consisting of lutein, zeaxanthin, and fatty acid esters thereof, and a component (C): a glycerol fatty acid ester (s), wherein the glycerol fatty acid ester (s) is a monoglyceride and/or a diglyceride containing a C12-C24 saturated fatty acid as a constituent fatty acid.
[2] The oral composition according to the clause [1], wherein the oral composition has a weight ratio of an amount of the component (C) to a sum of amounts of the component (A) (in terms of arachidonic acid) and the component (B) (in terms of free form) ((C)/((A)+(B))) of 0.40 to 20.
[3] The oral composition according to the clause [1] or [2], wherein the arachidonic acid-class compound is at least one compound selected from the group consisting of arachidonic acid, a salt thereof, and a compound containing arachidonic acid as a constituent fatty acid.
[4] The oral composition according to any one of the clauses [1] to [3], wherein the component (C) is contained in an amount of 4 to 25% by weight.
[5] The oral composition according to any one of the clauses [1] to [4], wherein the oral composition is to be charged into a soft capsule.
[6]A soft capsule containing: a filling containing the oral composition according to any one of the clauses [1] to [5]; and a coating.
[7]A method for producing an oral composition, the method including: mixing a component (A): an arachidonic acid-class compound; a component (B): at least one compound selected from the group consisting of lutein, zeaxanthin, and fatty acid esters thereof, and a component (C): a glycerol fatty acid ester (s), wherein the glycerol fatty acid ester (s) is a monoglyceride and/or a diglyceride containing a C12-C24 saturated fatty acid as a constituent fatty acid.

### Advantageous Effects of Invention

The present invention can provide an oral composition that contains an arachidonic acid-class compound and at least one compound selected from the group consisting of lutein, zeaxanthin, and fatty acid esters thereof and that is less prone to separation.

### DESCRIPTION OF EMBODIMENTS

An oral composition of the present invention contains: a component (A): an arachidonic acid-class compound; a component (B): at least one compound selected from the group consisting of lutein, zeaxanthin, and fatty acid esters thereof; and a component (C): a glycerol fatty acid ester (s).

### (Component (A): Arachidonic Acid-Class Compound)

The arachidonic acid-class compound may be any of arachidonic acid (free arachidonic acid), a salt thereof, and a derivative thereof. Examples of the salt of arachidonic acid include a calcium salt and a sodium salt. A usable derivative of arachidonic acid may be, for example, a compound having a structure in which arachidonic acid is bonded or a compound from which arachidonic acid can be isolated by hydrolysis. Examples of the compound from which arachidonic acid can be isolated by hydrolysis include compounds containing arachidonic acid as a constituent fatty acid. One arachidonic acid-class compound may be used alone, or two or more thereof may be used in combination.

The arachidonic acid-class compound for use in the present invention is preferably a compound selected from the group consisting of free arachidonic acid, its salt, and a compound containing arachidonic acid as a constituent fatty acid.

The compound containing arachidonic acid as a constituent fatty acid is preferably a compound selected from the group consisting of a triglyceride in which at least one or each constituent fatty acid is arachidonic acid, a diglyceride in which at least one or each constituent fatty acid is arachidonic acid, a monoglyceride in which a constituent fatty acid is arachidonic acid, a phospholipid in which at least one or each constituent fatty acid is arachidonic acid, a glycolipid in which at least one or each constituent fatty acid is arachidonic acid, and an alcohol ester of arachidonic acid.

Of these, the arachidonic acid-class compound is more preferably a triglyceride, diglyceride, or phospholipid in which at least one or each constituent fatty acid is arachidonic acid, still more preferably a triglyceride or diglyceride in which at least one or each constituent fatty acid is arachidonic acid, particularly preferably a triglyceride in which at least one or each constituent fatty acid is arachidonic acid.

The arachidonic acid-class compound for use in the present invention is not limited in any way by its form or production method, for example. A chemically synthesized product may be used, or a product derived from a natural product or a product derived from a genetically modified organism (e.g., Escherichia coli, yeast, plants) may be used. Examples of the product derived from a natural product include products derived from those capable of producing an arachidonic acid-class compound, such as fish and shellfish, algae, microorganisms, and plants (e.g., Marchantia polymorpha).

Of these, the arachidonic acid-class compound is preferably one derived from a microorganism. Examples of the microorganisms include those belonging to the genera Mortierella, Conidiobolus, Pythium, Phytophthora, Penicillium, Cladosporium, Mucor, Fusarium, Aspergillus, Rhodotorula, Entomophthora, Echinosporangium, and Saprolegnia.

The arachidonic acid-class compound used may be one extracted and/or purified from a raw material such as a natural product containing an arachidonic acid-class compound; alternatively, this raw material may be used as it is so that the oral composition of the present invention is made to contain an arachidonic acid-class compound. Examples of the raw material containing an arachidonic acid-class compound include edible fats and oils such as fats and oils produced by microorganisms as well as fats and oils extracted from eggs, meat, or marine products. Any method may be used to produce an arachidonic acid-class compound using a microorganism. Any known method, for example, the method disclosed in Patent Literature 1, can be used. In an embodiment, the oral composition of the present invention may contain a fat or oil, and the arachidonic acid-class compound may be part of the fat or oil.

### (Component (B): Lutein, Zeaxanthin, and Fatty Acid Esters Thereof)

The oral composition of the present invention contains at least one compound selected from the group consisting of lutein, zeaxanthin, and fatty acid esters thereof Lutein and zeaxanthin are types of carotenoid. The component (B) may include one or two or more of lutein (also referred to as free lutein or free form lutein), zeaxanthin (also referred to as free zeaxanthin or free form zeaxanthin), fatty acid esters of lutein, and fatty acid esters of zeaxanthin. For example, lutein, zeaxanthin, and fatty acid esters thereof can be prepared by extraction from plants. Lutein, zeaxanthin, and fatty acid esters thereof are contained, for example, in green and yellow vegetables such as spinach, kale, broccoli, squash, carrot, and paprika; fruits such as orange, peach, papaya, prune, mango, avocado, raspberry, and rose hip; and marigold petals.

Fatty acid esters of lutein are fatty acid esters in each of which one hydroxy group of lutein is coupled with a fatty acid or two hydroxy groups thereof are coupled with fatty acids. Examples of the fatty acids in the fatty acid esters of lutein include C4-C20 fatty acids, with C8-C18 fatty acids being preferred. The oral composition may contain one fatty acid ester of lutein or two or more fatty acid esters of lutein. Hydrolyzing a fatty acid ester of lutein can produce lutein.

Fatty acid esters of zeaxanthin are fatty acid esters in each of which one hydroxy group of zeaxanthin is coupled with a fatty acid or two hydroxy groups thereof are coupled with fatty acids. Examples of the fatty acids in the fatty acid esters of zeaxanthin include C4-C20 fatty acids, with C8-C18 fatty acids being preferred. The oral composition may contain one fatty acid ester of zeaxanthin or two or more fatty acid esters of zeaxanthin. Hydrolyzing a fatty acid ester of zeaxanthin can produce zeaxanthin.

In the present invention, as long as the effect of the present invention is achieved, the oral composition of the present invention may be made to contain any plant-derived material rich in at least one selected from the group consisting of lutein, fatty acid esters of lutein, zeaxanthin, and fatty acid esters of zeaxanthin, for example. Examples of the plant-derived material include plant-extracted fats and oils.

Lutein, zeaxanthin, and fatty acid esters thereof may be commercially available products.

The oral composition of the present invention preferably contains free lutein. The oral composition of the present invention preferably contains free zeaxanthin. The oral composition of the present invention preferably contains lutein and/or zeaxanthin as the component (B).

The oral composition of the present invention may contain only lutein and/or a fatty acid ester thereof or may contain only zeaxanthin and/or a fatty acid ester thereof as the component (B), and preferably contains lutein and zeaxanthin.

In the case where the oral composition of the present invention contains lutein and zeaxanthin, the ratio of lutein and zeaxanthin is not limited, and the weight ratio of lutein and zeaxanthin (lutein:zeaxanthin) is preferably 20:1 to 1:1, more preferably 10:1 to 2:1.

### (Component (C): Glycerol Fatty Acid Ester (s))

The oral composition of the present invention contains a glycerol fatty acid ester (s).

The glycerol fatty acid ester (s) is a monoglyceride and/or a diglyceride containing a C12-C24 saturated fatty acid as a constituent fatty acid. The presence of the glycerol fatty acid ester (s) allows the oral composition of the present invention to be less prone to separation.

In the present invention, the glycerol fatty acid ester (s) is a monoglyceride containing a C12-C24 saturated fatty acid as a constituent fatty acid and/or a diglyceride containing C12-C24 saturated fatty acids as constituent fatty acids.

The monoglyceride containing a C12-C24 saturated fatty acid as a constituent fatty acid is a compound in which a C12-C24 saturated fatty acid is ester-bonded to one of the three hydroxy groups of glycerol. The diglyceride containing C12-C24 saturated fatty acids as constituent fatty acids is a compound (glycerol di-fatty acid ester) in which C12-C24 saturated fatty acids are ester-bonded to two of the three hydroxy groups of glycerol. Herein, the “glycerol fatty acid ester” is a fatty acid monoglyceride and/or a fatty acid diglyceride.

Examples of the C12-C24 saturated fatty acid include lauric acid, myristic acid, pentadecylic acid, palmitic acid, margaric acid, stearic acid, arachidic acid, behenic acid, and lignoceric acid. The C12-C24 saturated fatty acid is preferably a C12-C22 saturated fatty acid. Specific examples of the monoglyceride or the diglyceride containing a C12-C24 saturated fatty acid as a constituent fatty acid include lauric acid monoglyceride, lauric acid diglyceride, palmitic acid monoglyceride, palmitic acid diglyceride, stearic acid monoglyceride, stearic acid diglyceride, behenic acid monoglyceride, and behenic acid diglyceride.

In the glycerol fatty acid ester (s), the constituent fatty acid thereof may include one or two or more selected from C12-C24 saturated fatty acids. The glycerol fatty acid ester (s) can also be referred to as a monoglyceride and/or a diglyceride in which the constituent fatty acid thereof include one or two or more selected from C12-C24 saturated fatty acids.

The glycerol fatty acid ester (s) is a monoglyceride and/or a diglyceride containing a C12-C24 saturated fatty acid as a constituent fatty acid. The proportion of the C12-C22 saturated fatty acid among the constituent fatty acids is preferably 90% by weight or more, more preferably 95% by weight or more, still more preferably 98% by weight or more.

The glycerol fatty acid ester (s) may include one or two or more glycerol fatty acid esters. In the case where the oral composition of the present invention contains two or more glycerol fatty acid esters as glycerol fatty acid esters (s), the combination thereof is not limited. For example, the presence of two or more glycerol fatty acid ester (s) in which the constituent fatty acids have different carbon numbers allows the oral composition of the present invention to be less prone to separation. The combination of two or more glycerol fatty acid ester (s) is preferably, for example, a combination of palmitic acid monoglyceride and stearic acid monoglyceride or a combination of stearic acid monoglyceride, stearic acid diglyceride, behenic acid monoglyceride, and behenic acid diglyceride.

The monoglyceride and/or the diglyceride containing a C12-C24 saturated fatty acid as a constituent fatty acid can be synthesized by a known method. The glycerol fatty acid ester (s) is generally produced by an esterification reaction between a saturated fatty acid and glycerol. The component (C) may be a commercially available emulsifier containing the glycerol fatty acid ester (s) as a main component. In the case of using a commercially available emulsifier, the emulsifier preferably contains the glycerol fatty acid ester (s) in an amount of more than 50% by weight. The amount of the glycerol fatty acid ester (s) in the emulsifier is more preferably 60% by weight or more, still more preferably 70% by weight or more, yet still more preferably 80% by weight or more, particularly preferably 90% by weight or more. In an embodiment, the amount of the glycerol fatty acid ester (s) in the emulsifier is preferably more than 50% by weight and 100% by weight or less, more preferably 60 to 100% by weight, still more preferably 70 to 100% by weight, yet still more preferably 80 to 100% by weight, particularly preferably 90 to 100% by weight, particularly preferably 95 to 100% by weight, particularly preferably 98 to 100% by weight, most preferably 99 to 100% by weight. In the present invention, the emulsifier is preferably one containing no unsaturated fatty acids as a constituent fatty acid.

As long as the effect of the present invention is not impaired, the oral composition of the present invention may contain a glycerol fatty acid ester other than the glycerol fatty acid ester (s) (referred to as a different glycerol fatty acid ester). The different glycerol fatty acid ester is preferably one containing no unsaturated fatty acids as a constituent fatty acid, for example.

In the oral composition of the present invention, the proportion of the C12-C24 saturated fatty acid in all of the constituent fatty acids of glycerol fatty acid esters including the glycerol fatty acid ester (s) is preferably 90 to 100% by weight, more preferably 95 to 100% by weight, still more preferably 98 to 100% by weight, particularly preferably 99 to 100% by weight. In an embodiment, in the case where the oral composition of the present invention contains the glycerol fatty acid ester (s) and a different glycerol fatty acid ester, the proportion of the C12-C24 saturated fatty acid in all of the constituent fatty acids of glycerol fatty acid esters including the glycerol fatty acid ester (s) is preferably 90% by weight or more and less than 100% by weight, more preferably 95% by weight or more and less than 100% by weight, still more preferably 98% by weight or more and less than 100% by weight, particularly preferably 99% by weight or more and less than 100% by weight.

Examples of a commercially available emulsifier that can be used in the present invention and that contains the glycerol fatty acid ester (s) as a main component include RIKEMAL S-100, RIKEMAL S-100P, RIKEMAL S-100A, RIKEMAL H-100, POEM V-100, POEM PV-100 (main component of all of the above: stearic acid monoglyceride), RIKEMAL B-100 (main component: behenic acid monoglyceride), and POEM M-300 (main component: lauric acid monoglyceride) (all of the above are available from Riken Vitamin Co., Ltd.).

The number of constituent fatty acids and unsaturated bonds of the constituent fatty acids of a glycerol fatty acid ester can be determined by gas chromatography (GC), for example.

The amount of the component (A): arachidonic acid-class compound (in terms of arachidonic acid) contained in the oral composition of the present invention is not limited and, for example, preferably 0.5% by weight or more, more preferably 1.0% by weight or more, still more preferably 1.5% by weight or more, while preferably 50% by weight or less, more preferably 45% by weight or less, still more preferably 40% by weight or less, particularly preferably 35% by weight or less in the oral composition. In an embodiment, the amount of the arachidonic acid-class compound (in terms of arachidonic acid) is preferably 0.5 to 50% by weight, more preferably 1.0 to 45% by weight, still more preferably 1.0 to 40% by weight, particularly preferably 1.5 to 35% by weight in the oral composition. The arachidonic acid-class compound contained in an amount within this range allows the oral composition of the present invention to be less prone to separation. In the case where two or more arachidonic acid-class compounds are contained, the amount refers to the total amount thereof. In the case where the arachidonic acid-class compound is not free arachidonic acid, arachidonic acid is liberated by saponification treatment or enzyme treatment, and the amount is calculated in terms of free arachidonic acid. The amount of arachidonic acid can be measured by GC.

In the case where the oral composition of the present invention contains a docosahexaenoic acid-class compound and an eicosapentaenoic acid-class compound to be described below, the amount of the arachidonic acid-class compound (in terms of arachidonic acid) may be 4 to 32% by weight in the oral composition.

The total amount of the component (B): lutein, zeaxanthin, and fatty acid esters thereof contained in the oral composition of the present invention is not limited and can be set according to the form thereof, for example.

The total amount of the component (B) (in terms of free form) in the oral composition of the present invention is, for example, preferably 0.01% by weight or more, more preferably 0.1% by weight or more, while preferably 14% by weight or less, more preferably 11% by weight or less in the oral composition. In an embodiment, the total amount of the component (B) (in terms of free form) is preferably 0.01 to 14% by weight, more preferably 0.1 to 110% by weight in the oral composition. The component (B) contained in a total amount within this range can further reduce separation of the oral composition of the present invention. Further, the oral composition of the present invention can efficiently exert the physiological functions of lutein, zeaxanthin, and/or fatty acid esters thereof.

For the component (B), the term “in terms of free form” means that the amount of lutein and/or a fatty acid ester thereof is expressed in terms of lutein and that the amount of zeaxanthin and/or a fatty acid ester thereof is expressed in terms of zeaxanthin.

In the case where the component (B) is lutein, the expression “amount in terms of lutein” or a similar expression means the amount of lutein; in the case where the component (B) is a fatty acid ester of lutein, the term or a similar expression means a value obtained by multiplying the number of moles of the ester by the molecular weight of lutein.

In the case where the component (B) is zeaxanthin, the expression “amount in terms of zeaxanthin” or a similar expression means the amount of zeaxanthin; in the case where the component (B) is a fatty acid ester of zeaxanthin, the term or a similar expression means a value obtained by multiplying the number of moles of the ester by the molecular weight of zeaxanthin.

The amounts of lutein, zeaxanthin, and fatty acid esters thereof can be determined by HPLC (high performance liquid chromatography).

The amount of the component (C): glycerol fatty acid ester (s) in the oral composition of the present invention is preferably 4% by weight or more, more preferably 10% by weight or more, still more preferably 15% by weight or more relative to the whole amount of the oral composition. The amount is preferably 25% by weight or less, more preferably 22% by weight or less. In an embodiment, the amount of the component (C) is preferably 4 to 25% by weight, more preferably 10 to 22% by weight, still more preferably 15 to 22% by weight in the oral composition of the present invention. The component (C) in an amount within this range relative to the whole amount of the oral composition allows the oral composition to be stable, less prone to separation. Also, 25% by weight or less of the component (C) relative to the whole amount of the oral composition allows the oral composition to have a viscosity that is not excessively high, which leads to good workability when the oral composition is charged into a soft capsule, for example. The amount of the component (C) can be determined by GC or HPLC.

The oral composition of the present invention has a weight ratio of the amount of the component (C) to the sum of the amounts of the component (A) (in terms of arachidonic acid) and the component (B) (in terms of free form) ((C)/((A)+(B))) of preferably 0.40 to 20. The presence of the component (A), the component (B), and the component (C) in this weight ratio allows the oral composition of the present invention to be much less prone to separation. The weight ratio is more preferably 0.45 or more, still more preferably 0.50 or more, while more preferably 18 or less. In an embodiment, the weight ratio is more preferably 0.45 to 18, still more preferably 0.50 to 18.

The oral composition of the present invention has a weight ratio of the component (A) (in terms of arachidonic acid) to the component (B) (in terms of free form) ((A)/(B)) of preferably 0.1 or more, more preferably 0.5 or more, still more preferably 1.5 or more, particularly preferably 1.9 or more, while preferably 40 or less, more preferably 35 or less. In an embodiment, the weight ratio of the component (A) (in terms of arachidonic acid) to the component (B) (in terms of free form) ((A)/(B)) is preferably 0.1 to 40, more preferably 0.5 to 40, still more preferably 1.5 to 35, particularly preferably 1.9 to 35.

The oral composition of the present invention may be in any form such as a solid (e.g., powder, granule, or tablet), liquid, or paste. The oral composition of the present invention is preferably liquid at 25° C. The oral composition of the present invention is preferably a composition in which the components are not separated from each other.

Arachidonic acid-class compounds as well as lutein, zeaxanthin, and fatty acid esters thereof are compounds that are found in natural products and food and beverages and that have been consumed. Thus, for example, daily ingestion of an arachidonic acid-class compound as well as lutein, zeaxanthin, and a fatty acid ester thereof is less likely to cause problems in terms of safety. The present invention can provide a highly safe and easy-to-ingest oral composition.

Arachidonic acid-class compounds are known to exert various health functions such as an effect of improving cognitive function and an effect of regulating immune function.

Since the oral composition of the present invention contains an arachidonic acid-class compound, the oral composition can also be used to obtain the above effects exerted by the arachidonic acid-class compound.

Lutein, zeaxanthin, and fatty acid esters thereof are known to have, for example, the neuroprotective effect owing to the anti-oxidative effect in the cranial nervous system.

Therefore, the oral composition of the present invention can also be used to obtain the above effects exerted by at least one selected from the group consisting of lutein, zeaxanthin, and fatty acid esters thereof.

The oral composition of the present invention is applicable for therapeutic use (medical use) and non-therapeutic use (non-medical use). The “non-therapeutic” is a concept that does not include medical activities, i.e., a concept that does not include methods of surgery, therapy, or diagnosis of humans.

The oral composition of the present invention can be provided in the form of a food or beverage, a pharmaceutical product, a quasi-pharmaceutical product, feed, or the like. The oral composition of the present invention may be a material or preparation to be added to a food or beverage, a pharmaceutical product, a quasi-pharmaceutical product, or feed, for example. The oral composition refers to a composition that can be ingested orally.

Specific examples of the oral composition of the present invention include a food and beverage, an oral pharmaceutical product, an oral quasi-pharmaceutical product, and feed. A food or beverage or an oral pharmaceutical product is preferred, and a food or beverage is more preferred.

The oral composition of the present invention may contain optional additives and optional components in addition to the components (A) to (C) as long as the effect of the present invention is not impaired. Such additives and components can be selected according to the form of the oral composition, for example, and the oral composition may contain any additives and components that can be commonly used for oral compositions such as food and beverages, pharmaceutical products, quasi-pharmaceutical products, and feed. In the case where the oral composition of the present invention is provided as a food or beverage, a pharmaceutical product, a quasi-pharmaceutical product, feed, or the like, any common method can be used for production.

Examples of the optional components include oil-based components. Examples of the oil-based components include highly unsaturated fatty acids other than the above components (A) to (C) and fats and oils.

Examples of the oil-based components include a docosahexaenoic acid-class compound, an eicosapentaenoic acid-class compound, and edible fats and oils.

The oral composition of the present invention preferably further contains a docosahexaenoic acid-class compound and an eicosapentaenoic acid-class compound. In the oral composition of the present invention, the components are less prone to separate even when the oral composition contains oil-based components such as a docosahexaenoic acid-class compound and an eicosapentaenoic acid-class compound.

The docosahexaenoic acid-class compound may be any of docosahexaenoic acid (free DHA), a salt thereof, and a derivative thereof. The derivative of docosahexaenoic acid may be, for example, a compound that has a structure containing docosahexaenoic acid bonded thereto or a compound that allows docosahexaenoic acid to be dissociable by hydrolysis. An example of the compound that allows docosahexaenoic acid to be dissociable by hydrolysis is a compound containing docosahexaenoic acid as a constituent fatty acid. The oral composition of the present invention may contain one docosahexaenoic acid-class compound or two or more docosahexaenoic acid-class compounds.

The docosahexaenoic acid-class compound for use in the present invention is preferably at least one selected from the group consisting of free docosahexaenoic acid, a salt thereof, and a compound containing docosahexaenoic acid as a constituent fatty acid.

Examples of the salt of docosahexaenoic acid include a calcium salt and a sodium salt.

The compound containing docosahexaenoic acid as a constituent fatty acid is preferably a compound selected from the group consisting of a triglyceride in which at least one or each constituent fatty acid is docosahexaenoic acid, a diglyceride in which at least one or each constituent fatty acid is docosahexaenoic acid, a monoglyceride in which a constituent fatty acid is docosahexaenoic acid, a phospholipid in which at least one or each constituent fatty acid is docosahexaenoic acid, a glycolipid in which at least one or each constituent fatty acid is docosahexaenoic acid, and an alcohol ester of docosahexaenoic acid.

Of these, the docosahexaenoic acid-class compound is more preferably a triglyceride, diglyceride, or phospholipid in which at least one or each constituent fatty acid is docosahexaenoic acid, still more preferably a triglyceride or diglyceride in which at least one or each constituent fatty acid is docosahexaenoic acid, particularly preferably a triglyceride in which at least one or each constituent fatty acid is docosahexaenoic acid.

The docosahexaenoic acid-class compound for use in the present invention is not limited in any way by its form or production method, for example. A chemically synthesized product may be used, or a product derived from a natural product or a product derived from a genetically modified organism (e.g., Escherichia co/i, yeast, plants) may be used. Examples of the product derived from a natural product include products derived from those capable of producing DHA, such as fish and shellfish, algae, fungi (e.g., bacteria of the genus Colwellia, labyrinthulids), and plants (e.g., Marchantia polymorpha). Of these, the docosahexaenoic acid-class compound is preferably one derived from fish or shellfish.

The docosahexaenoic acid-class compound used may be one extracted and/or purified from a raw material such as a natural product containing a docosahexaenoic acid-class compound; alternatively, this raw material may be used as it is so that the composition is made to contain a docosahexaenoic acid-class compound. Examples of the raw material containing a docosahexaenoic acid-class compound include edible fats and oils such as fish oil as well as fats and oils extracted from meat or milk. The fish oil containing a docosahexaenoic acid-class compound is preferably a concentrated fish oil in which the proportion of the docosahexaenoic acid-class compound (in terms of docosahexaenoic acid) contained is 5% by weight or more, preferably 10% by weight or more, more preferably 50% by weight or more. For example, a docosahexaenoic acid-class compound extracted and/or purified from fish oil containing a docosahexaenoic acid-class compound may be used, or this fish oil may be used as it is. In an embodiment, the oral composition of the present invention may contain fish oil, and the docosahexaenoic acid-class compound may be part of this fish oil.

The eicosapentaenoic acid-class compound may be any of eicosapentaenoic acid (free eicosapentaenoic acid; hereinafter, also referred to as EPA), a salt thereof, and a derivative thereof. One eicosapentaenoic acid-class compound may be used alone, or two or more eicosapentaenoic acid-class compounds may be used in combination. The derivative of eicosapentaenoic acid may be, for example, a compound that has a structure containing eicosapentaenoic acid bonded thereto or a compound that allows eicosapentaenoic acid to be dissociable by hydrolysis. An example of the compound that allows eicosapentaenoic acid to be dissociable by hydrolysis is a compound containing eicosapentaenoic acid as a constituent fatty acid.

The eicosapentaenoic acid-class compound is preferably at least one selected from the group consisting of free eicosapentaenoic acid, a salt thereof, and a compound containing eicosapentaenoic acid as a constituent fatty acid.

Examples of the salt of eicosapentaenoic acid include a calcium salt and a sodium salt.

The compound containing eicosapentaenoic acid as a constituent fatty acid is preferably a compound selected from the group consisting of a triglyceride in which at least one or each constituent fatty acid is eicosapentaenoic acid, a diglyceride in which at least one or each constituent fatty acid is eicosapentaenoic acid, a monoglyceride in which a constituent fatty acid is eicosapentaenoic acid, a phospholipid in which at least one or each constituent fatty acid is eicosapentaenoic acid, a glycolipid in which at least one or each constituent fatty acid is eicosapentaenoic acid, and an alcohol ester of eicosapentaenoic acid.

Of these, the eicosapentaenoic acid-class compound is more preferably a triglyceride, diglyceride, or phospholipid in which at least one or each constituent fatty acid is eicosapentaenoic acid, still more preferably a triglyceride or diglyceride in which at least one or each constituent fatty acid is eicosapentaenoic acid, particularly preferably a triglyceride in which at least one or each constituent fatty acid is eicosapentaenoic acid.

The eicosapentaenoic acid-class compound is not limited in any way by its form or production method, for example. A chemically synthesized product may be used, or a product derived from a natural product or a product derived from a genetically modified organism (e.g., Escherichia coli, yeast, plants) may be used. Examples of the product derived from a natural product include products derived from those capable of producing EPA, such as fish and shellfish, algae, fungi (e.g., labyrinthulids), and plants (e.g., Marchantia polymorpha). Of these, the eicosapentaenoic acid-class compound is preferably one derived from fish or shellfish.

The eicosapentaenoic acid-class compound used may be one extracted and/or purified from a raw material such as a natural product containing an eicosapentaenoic acid-class compound; alternatively, this raw material may be used as it is so that the composition is made to contain an eicosapentaenoic acid-class compound. Examples of the raw material containing an eicosapentaenoic acid-class compound include edible fats and oils such as fish oil as well as fats and oils extracted from eggs, meat, milk, or algae. The fish oil containing an eicosapentaenoic acid-class compound is preferably a concentrated fish oil in which the proportion of the eicosapentaenoic acid-class compound (in terms of eicosapentaenoic acid) contained is 5% by weight or more, preferably 10% by weight or more, more preferably 20% by weight or more. For example, an eicosapentaenoic acid-class compound extracted and/or purified from fish oil containing an eicosapentaenoic acid-class compound may be used, or this fish oil may be used as it is.

In the case where the oral composition of the present invention contains free DHA, the expression “amount in terms of docosahexaenoic acid” or a similar expression means the amount of free DHA; in the case where a docosahexaenoic acid-class compound other than free DHA is contained, the term or a similar expression means the amount in terms of free DHA, which is obtained by liberating DHA from a compound containing DHA as a constituent fatty acid by saponification treatment or enzyme treatment.

In the case where the oral composition of the present invention contains free EPA, the expression “amount in terms of eicosapentaenoic acid” or a similar expression means the amount of free EPA; in the case where an eicosapentaenoic acid-class compound other than free EPA is contained, the term or a similar expression means the amount in terms of free EPA, which is obtained by liberating EPA from a compound containing EPA as a constituent fatty acid by saponification treatment or enzyme treatment.

For the oil-based component, the edible fats and oils are any fats and oils that can be orally ingested by humans, such as edible fats and oils that are liquid at room temperature (25° C.). Examples of these edible fats and oils include vegetable fats and oils such as olive oil, sesame oil, rice germ oil, safflower oil, soybean oil, corn oil, rapeseed oil, palm oil, palm olein, palm kernel oil, sunflower oil, grape oil, cottonseed oil, coconut oil, and peanut oil; medium-chain fatty acid triglycerides; squalene; and fish oil. The fish oil may be one containing a docosahexaenoic acid-class compound and/or an eicosapentaenoic acid-class compound, or may be one not containing a docosahexaenoic acid-class compound or an eicosapentaenoic acid-class compound.

In the oral composition of the present invention, one oil-based component may be used alone or two or more oil-based components may be used in combination.

The oil-based component is preferably a docosahexaenoic acid-class compound or an eicosapentaenoic acid-class compound.

The oral composition of the present invention preferably contains fish oil containing a docosahexaenoic acid-class compound, fish oil containing an eicosapentaenoic acid-class compound, and/or fish oil containing a docosahexaenoic acid-class compound and an eicosapentaenoic acid-class compound.

In the case where the oral composition of the present invention contains the above oil-based component, the amount of the oil-based component is preferably, but not limited to, 10% by weight or more relative to the whole amount of the oral composition. The oil-based component contained in an amount within this range allows the oral composition of the present invention to be less prone to separation. The amount of the oil-based component in the oral composition can be set as appropriate according to the intended use and the form, and may be, for example, 20% by weight or more or 40% by weight or more. The amount may be, for example, 90% by weight or less, 80% by weight or less, or 70% by weight or less. In an embodiment, the amount of the oil-based component may be 10 to 90% by weight, 20 to 80% by weight, or 40 to 70% by weight in the oral composition of the present invention.

The composition and the amount of the oil-based component can be determined by gas chromatography (GC).

In the case where the oral composition of the present invention contains a docosahexaenoic acid-class compound, the total amount of the docosahexaenoic acid-class compound in the oral composition of the present invention is, for example, preferably 1% by weight or more, more preferably 10% by weight or more, while preferably 80% by weight or less in the oral composition. In an embodiment, the total amount of the docosahexaenoic acid-class compound is preferably 1 to 80% by weight, more preferably 10 to 80% by weight in the oral composition.

In the case where the oral composition of the present invention contains an eicosapentaenoic acid-class compound, the total amount of the eicosapentaenoic acid-class compound in the oral composition of the present invention is, for example, preferably 1% by weight or more, more preferably 3% by weight or more, while preferably 40% by weight or less, more preferably 30% by weight or less in the oral composition. In an embodiment, the total amount of the eicosapentaenoic acid-class compound is preferably 1 to 40% by weight, more preferably 3 to 30% by weight in the oral composition.

For example, in the case where the oral composition of the present invention is provided as a food or beverage, the components (A) to (C) may be compounded with any component usable in foods and beverages (e.g., food materials and optional food additives) to provide a variety of foods and beverages. Non-limiting examples of the foods and beverages include general foods and beverages, health foods, dietary supplements, health beverages, foods with function claims, foods for specified health uses, and foods and beverages for the sick. The health foods, dietary supplements, foods with function claims, foods for specified health uses, and the like can be used in various forms of preparations such as fine granules, tablets, granules, powders, capsules, soft capsules, chewable tablets, dry syrups, syrups, liquid agents, beverages, energy drinks, and liquid foods.

In the case where the oral composition of the present invention is provided as a pharmaceutical product or a quasi-pharmaceutical product, for example, the components (A) to (C) may be compounded with any component such as a pharmaceutically acceptable carrier and an additive that may be added optionally to provide pharmaceutical products or quasi-pharmaceutical products in various dosage forms. These components such as a carrier and an additive are pharmaceutically acceptable ones usable for pharmaceutical products and quasi-pharmaceutical products, and may include one or two or more selected from vehicles, binders, disintegrants, lubricants, antioxidants, colorants, and the like. Examples of dosage forms for oral administration of a pharmaceutical product or a quasi-pharmaceutical product include liquid agents, tablets, powders, fine granules, granules, sugar-coated tablets, capsules, soft capsules, suspensions, emulsions, and chewable tablets.

In the case where the oral composition of the present invention is provided as feed, a mixture of the components (A) to (C) is simply added to feed. The feed includes feed additives. Examples of the feed include feed for farm animals such as cows, pigs, chickens, sheep, and horses; feed for small animals such as rabbits, rats, and mice; and pet food for animals such as dogs, cats, and birds.

The subject (which may also be referred to as the administration subject) who ingests the oral composition of the present invention or to whom the composition is administered is not limited, and is preferably a human or non-human mammal, more preferably a human.

In the oral composition of the present invention, the amount of the arachidonic acid-class compound in terms of arachidonic acid per adult daily intake is preferably 10 to 500 mg, more preferably 20 to 400 mg, still more preferably 30 to 300 mg.

In the oral composition of the present invention, the amount of lutein and/or a fatty acid ester thereof in terms of lutein per adult daily intake is preferably 0.5 to 200 mg, more preferably 1 to 100 mg, still more preferably 3 to 80 mg.

In the oral composition of the present invention, the amount of zeaxanthin and/or a fatty acid ester thereof in terms of zeaxanthin per adult daily intake is preferably 0.1 to 50 mg, more preferably 0.1 to 30 mg, still more preferably 0.5 to 20 mg.

### <Method for Producing Oral Composition>

The oral composition of the present invention can be produced by, for example, a method including mixing the component (A), the component (B), and the component (C).

The method for producing an oral composition of the present invention includes: mixing a component (A): an arachidonic acid-class compound; a component (B): at least one compound selected from the group consisting of lutein, zeaxanthin, and fatty acid esters thereof; and a component (C): a glycerol fatty acid ester (s), wherein the glycerol fatty acid ester (s) is a monoglyceride and/or a diglyceride containing a C12-C24 saturated fatty acid as a constituent fatty acid.

The arachidonic acid-class compound, at least one compound selected from the group consisting of lutein, zeaxanthin, and fatty acid esters thereof, and glycerol fatty acid ester (s) may be mixed in any order and with any device. Preferably, the glycerol fatty acid ester (s) of the component (C) is heated to 70 to 80° C. for dissolution, and then a mixture of the component (A) and the component (B) is added to the glycerol fatty acid ester (s), followed by mixing and stirring, whereby the oral composition of the present invention can be produced. The device for heating may be a water bath, for example. The device for the mixing and stirring may be a high-speed stirrer or a high-speed grinder such as a biomixer, a homo-jetter, or a colloid mill, for example.

Preferred embodiments of the amounts, the weight ratio, and the like of the components in the oral composition are as described above. The components are preferably compounded so that the amounts of the components, for example, in the oral composition fall within the above respective ranges.

The oral composition of the present invention can be suitably used as a filling of a soft capsule. The oral composition of the present invention is suitable as a composition to be charged into a soft capsule, for example.

### <Soft Capsule>

The present invention also relates to a soft capsule containing a filling containing the oral composition and a coating.

In the soft capsule of the present invention, the filling containing the oral composition is charged into a soft capsule formed of a coating. The filling may contain a different component as long as it contains the oral composition. A base material for forming the coating may be a base material commonly used for coatings of soft capsules (e.g., a high molecular weight compound such as gelatin or starch).

### <Method for Producing Soft Capsule>

The oral composition can be provided as a soft capsule using a known soft capsule forming machine in a heated state, for example. A soft capsule can be produced according to production conditions of a soft capsule encapsulation device without limitation.

The numerical range defined by the lower limit and the upper limit herein, i.e., “the lower limit to the upper limit”, includes the lower limit and the upper limit. For example, the range defined by “1 to 2” means 1 or more and 2 or less, with 1 and 2 being included. Herein, the range may be any combination of any upper limit and any lower limit.

### EXAMPLES

The present invention is described in further detail below with reference to examples. The present invention is not limited to these examples.

### Example 1

### <Production of Oral Composition>

First, 23.5 g of an arachidonic acid-containing edible fat or oil derived from a microorganism (ARA-containing edible fat or oil) and 1.2 g of a marigold pigment raw material (containing 20% by weight or more of lutein and 4% by weight or more of zeaxanthin) were mixed in a beaker, whereby a liquid mixture was prepared. Next, in order to uniformly disperse the marigold pigment raw material in the liquid mixture, the liquid mixture was stirred with a stirrer at a number of revolutions of 200 rpm for 1 to 1.5 hours.

Next, 5.3 g of a glycerol fatty acid ester (C1) shown in Table 2 was dispensed into a polypropylene tube. With a water bath set to 75° C., the glycerol fatty acid ester (C1) was dissolved under heating.

The liquid mixture prepared above was then added to this polypropylene tube containing the glycerol fatty acid ester (C1) dissolved. At this time, the glycerol fatty acid ester (C1) was solidified due to a temperature decrease caused by the addition of the liquid mixture. Therefore, in order to redissolve the glycerol fatty acid ester (C1), a mixture of the liquid mixture and the glycerol fatty acid ester (C1) was heated in a water bath set to a temperature of 75° C. After the redissolution of the glycerol fatty acid ester (C1) was confirmed, the resultant mixture was stirred using a vortex mixer. Further, in order to uniformly dissolve the mixture, the mixture was heated again in a water bath set at a temperature of 75° C. After the uniform mixing of the mixture was visually confirmed, the polypropylene tube containing the mixture was taken out of the water bath and allowed to cool at room temperature. This was provided as an oral composition. Table 1 shows the amounts (g) of the raw materials compounded in the preparation of the oral composition, the proportions (% by weight) of the components in the oral composition, and the weight ratios of the components. Table 2 shows the composition and the number of unsaturated bonds of the glycerol fatty acid ester (C1). In Table 1 and Table 3 to be shown below, the weight proportion (% by weight) of the arachidonic acid (A) refers to the weight of the arachidonic acid-class compound contained in the oral composition in terms of arachidonic acid; the weight proportion of the lutein and zeaxanthin (B) refers to the sum of the weight of lutein and fatty acid esters thereof in terms of lutein and the weight of zeaxanthin and fatty acid esters thereof in terms of zeaxanthin.

### <Separation Evaluation>

After the oral composition was allowed to cool, the oral composition was dispensed from the polypropylene tube into a commercially available plastic cup using a dispensing spoon. The presence or absence of separation at this time was visually evaluated. The evaluation criteria are as follows. The results are shown in Table 2.
∘∘: Not separated.
∘: Hardly separated (when a small amount of the oral composition was sampled, liquid oozed out of the circumference thereof).
Δ: The oral composition partially separated.
x: The oral composition completely separated.

### Examples 2 to 6

Oral compositions according to Examples 2 to 6 were prepared in the same manner as in Example 1 except that the glycerol fatty acid ester (C1) was changed to glycerol fatty acid esters (C2) to (C6) shown in Table 2, and were subjected to the separation evaluation in the same manner as in Example 1. The results are shown in Table 2.

### Comparative Examples 1 to 10

Oral compositions according to Comparative Examples 1 to 10 were prepared in the same manner as in Example 1 except that the glycerol fatty acid ester (C1) was changed to (poly)glycerol fatty acid esters (C′1) to (C′10) shown in Table 2, and were subjected to the separation evaluation in the same manner as in Example 1. The results are shown in Table 2.

TABLE 1

| | | | Comparative |
|---|---|---|---|
| | | Examples | Examples |
| | | 1 to 6 | 1 to 10 |
| Amount | Arachidonic acid-containing edible fat or oil | 23.5 | 23.5 |
| (g) | Marigold pigment raw material | 1.2 | 1.2 |
| | (C) or (C′) in Table 2 | 5.3 | 5.3 |
| | Total | 30 | 30 |
| Proportion of | Arachidonic acid (A) | 30.69 | 30.69 |
| component | Lutein and zeaxanthin (B) | 1.02 | 1.02 |
| (wt %) | Glycerol fatty acid ester (C) | 17.53 | — |
| | (Poly)glycerol fatty acid ester (C′) | — | 17.53 |
| Weight ratio of | (C)/(A) + (B) | 0.55 | — |
| components | (A)/(B) | 30.09 | 30.09 |

TABLE 2

| | | | | Number of | |
|---|---|---|---|---|---|
| | | | | unsaturated | Separation |
| | | Main componnet | Category | bonds | evaluation |
| | Glycerol fatty | | | | |
| | acid ester (C) | | | | |
| Example 1 | (C1) | Hydrogenated rapeseed oil fatty | Fatty acid mono/diglyceride | 0 | ∘∘ |
| | | acid mono/diglyceride | | | |
| Example 2 | (C2) | Self-emulsifying stearic acid | Fatty acid mono/diglyceride | 0 | ∘ |
| | | mono/diglyceride | | | |
| Example 3 | (C3) | Lauric acid monoglyceride | Fatty acid monoglyceride | 0 | ∘∘ |
| Example 4 | (C4) | Hydrogenated palm oil | Fatty acid monoglyceride | 0 | ∘∘ |
| | | monoglyceride | | | |
| Example 5 | (C5) | Lauric acid monoglyceride | Fatty acid monoglyceride | 0 | ∘∘ |
| Example 6 | (C6) | Stearic acid monoglyceride | Fatty acid monoglyceride | 0 | ∘∘ |
| | | Palmitic acid monoglyceride | | | |
| | (Poly)glycerol fatty | | | | |
| | acid ester (C′) | | | | |
| Comparative Example 1 | (C′1) | Diglycerol mono/distearate | Polyglycerol fatty acid ester | 0 | Δ |
| Comparative Example 2 | (C′2) | Decaglycerol decastearate | Polyglycerol fatty acid ester | 0 | x |
| Comparative Example 3 | (C′3) | Oleic acid monoglyceride | Fatty acid monoglyceride | 1 | x |
| Comparative Example 4 | (C′4) | Diglycerol mono/dioleate | Polyglycerol fatty acid ester | 1 | x |
| Comparative Example 5 | (C′5) | Oleic acid mono/diglyceride | Fatty acid mono/diglyceride | 1 | x |
| Comparative Example 6 | (C′6) | Caprylic acid monoglyceride | Fatty acid monoglyceride | 0 | x |
| Comparative Example 7 | (C′7) | Decaglycerol decaoleate | Polyglycerol fatty acid ester | 1 | x |
| Comparative Example 8 | (C′8) | Decaglycerol pentaoleate | Polyglycerol fatty acid ester | 1 | x |
| Comparative Example 9 | (C′9) | Diglycerol monolaurate | Polyglycerol fatty acid ester | 0 | x |
| Comparative Example 10 | (C′10) | Glycerol citrate monooleate | Organic acid monoglyceride | 1 | x |

The “Number of unsaturated bonds” in Table 2 shows the number of unsaturated bonds in the constituent fatty acids of the glycerol fatty acid ester. The glycerol fatty acid esters (C1) and (C2) are each a mixture of a monoglyceride containing a C12-C24 saturated fatty acid as a constituent fatty acid and a diglyceride containing C12-C24 saturated fatty acids as constituent fatty acids. The glycerol fatty acid esters (C3) to (C6) each include a monoglyceride containing a C12-C24 saturated fatty acid as a constituent fatty acid. The main constituent fatty acids of the glycerol fatty acid ester (C1) were palmitic acid (number of carbon atoms: 16, 4.2% by weight), stearic acid (number of carbon atoms: 18, 41.9% by weight), arachidic acid (number of carbon atoms: 20, 8.8% by weight), and behenic acid (number of carbon atoms: 22, 43.5% by weight). Among the constituent fatty acids of the glycerol fatty acid ester (C1), the C12-C24 saturated fatty acids other than palmitic acid, stearic acid, arachidic acid, and behenic acid accounted for 1.5% by weight. The main constituent fatty acids of the glycerol fatty acid ester (C4) were palmitic acid (number of carbon atoms: 16, 43.0% by weight) and stearic acid (55.1% by weight). Among the constituent fatty acids of the glycerol fatty acid ester (C4), the C12-C24 saturated fatty acids other than palmitic acid and stearic acid accounted for about 1.9% by weight. Among the constituent fatty acids of the glycerol fatty acid ester (C6), myristic acid (number of carbon atoms: 14) accounted for 2.4% by weight, margaric acid (number of carbon atoms: 17) accounted for 1.1% by weight, arachidic acid (number of carbon atoms: 20) accounted for 1.4% by weight, stearic acid accounted for 28.4% by weight, and palmitic acid accounted for 65.3% by weight. Among the constituent fatty acids of the glycerol fatty acid ester (C6), the C12-C24 saturated fatty acids other than myristic acid, margaric acid, arachidic acid, palmitic acid, and stearic acid accounted for about 0.9% by weight. Analysis of the constituent fatty acids was outsourced to Japan Food Research Laboratories and performed by gas chromatography (flame ionization detector, FID).

For the glycerol fatty acid esters (C1) to (C6), C12-C22 saturated fatty acids accounted for 90% by weight or more of the constituent fatty acids.

As shown in Table 2, the oral compositions of Examples 1 to 6 did not separate or hardly separated. The oral compositions of Comparative Examples 1 to 10 completely separated or partially separated.

The main component of each of the glycerol fatty acid esters used in Examples 1 to 6 was a monoglyceride containing a C12-C24 saturated fatty acid as a constituent fatty acid and/or a diglyceride containing C12-C24 saturated fatty acids as constituent fatty acids.

In Comparative Examples 1, 2, 4, and 7 to 9, a polyglycerol fatty acid ester was used. In Comparative Example 6, a glycerol fatty acid ester containing a C8 constituent fatty acid was used. The (poly)glycerol fatty acid esters (C′) of Comparative Examples 3 to 5, 7, 8, and 10 contained an unsaturated fatty acid as a constituent fatty acid.

These results therefore have demonstrated that, in the composition containing arachidonic acid and lutein and/or zeaxanthin, the presence of a monoglyceride and/or a diglyceride containing a C12-C24 saturated fatty acid as a constituent fatty acid reduces separation of the composition.

### Examples 7 to 15 and Reference Example 1

### <Examination of Proportions of Components in Oral Composition>

Oral compositions were prepared with different proportions of arachidonic acid, lutein and/or zeaxanthin, and a glycerol fatty acid ester.

Oral compositions according to Examples 7 to 15 and Reference Example 1 were prepared in the same manner as in Example 1 except that the composition was changed to those shown in Table 3. The oral compositions obtained were subjected to the separation evaluation in the same manner as in Example 1. In Examples 7 to 15 and Reference Example 1, the total weight of the oral composition was made to be 30 g with safflower oil being added to compensate for the shortfall. The glycerol fatty acid ester used was the glycerol fatty acid ester (C6) of Example 6. The results are shown in Table 3.

TABLE 3

| | | | | | | | | | | | Reference |
|---|---|---|---|---|---|---|---|---|---|---|---|
| | | Exam- | Exam- | Exam- | Exam- | Exam- | Exam- | Exam- | Exam- | Exam- | Exam- |
| | | ple 7 | ple 8 | ple 9 | ple 10 | ple 11 | ple 12 | ple 13 | ple 14 | ple 15 | ple 1 |
| Amount | ARA-containing edible fat or oil | 1.5 | 3.3 | 6.0 | 9.3 | 18.3 | 6.0 | 6.0 | 6.0 | 6.0 | 0 |
| (g) | Marigold pigment raw material | 1.2 | 1.2 | 1.2 | 1.2 | 1.2 | 1.2 | 1.2 | 1.2 | 1.2 | 1.2 |
| | Glycerol fatty acid ester (C6) | 5.3 | 5.3 | 5.3 | 5.3 | 5.3 | 1.4 | 3.0 | 4.6 | 6.2 | 5.3 |
| | Edible fat or oil (safflower oil) | 22.1 | 20.3 | 17.6 | 14.3 | 5.3 | 21.4 | 19.8 | 18.3 | 16.6 | 23.6 |
| | Total | 30 | 30 | 30 | 30 | 30 | 30 | 30 | 30 | 30 | 30 |
| Proportion of | Arachidonic acid (A) | 1.94 | 4.29 | 7.82 | 12.13 | 23.88 | 7.82 | 7.82 | 7.82 | 7.82 | 0 |
| component | Lutein and zeaxanthin (B) | 1.02 | 1.02 | 1.02 | 1.02 | 1.02 | 1.02 | 1.02 | 1.02 | 1.02 | 1.02 |
| (wt %) | Glycerol fatty acid ester (C6) | 17.53 | 17.53 | 17.53 | 17.53 | 17.53 | 4.73 | 10.12 | 15.24 | 20.67 | 17.53 |
| Weight ratio of | (C)/(A) + (B) | 5.92 | 3.30 | 1.98 | 1.33 | 0.70 | 0.54 | 1.14 | 1.72 | 2.34 | 17.19 |
| components | (A)/(B) | 1.9 | 4.2 | 7.7 | 11.9 | 23.4 | 7.7 | 7.7 | 7.7 | 7.7 | 0 |

| | | | | | | | | | | |
|---|---|---|---|---|---|---|---|---|---|---|
| Separation evaluation | ◯◯ | ◯◯ | ◯◯ | ◯◯ | ◯◯ | ◯ | ◯ | ◯◯ | ◯◯ | ◯◯ |

As shown in Table 3, the oral compositions of Example 7 to 15 did not separate or hardly separated. In the oral compositions of Examples 7 to 15, the value of (glycerol fatty acid ester)/(sum of arachidonic acid, lutein, and zeaxanthin) was 0.40 to 20 and the emulsifier content was 4 to 25% by weight.

These results therefore have suggested that separation of the oral composition is reduced in the case where the value of (glycerol fatty acid ester)/(sum of arachidonic acid, lutein, and zeaxanthin) falls within the range of 0.40 to 20 and the emulsifier content falls within the range of 4 to 25% by weight in the oral composition. The oral composition of Reference Example 1 did not contain an arachidonic acid-class compound, but did not separate as with Examples 7 to 15.

### Examples 16 to 21 and Comparative Examples 11 to 20

### <Production of Oral Composition>

Oral compositions according to Examples 16 to 21 and Comparative Examples 11 to 20 were prepared and subjected to the separation evaluation as in Example 1 except that DHA- and EPA-containing refined fish oil was added in preparing a liquid mixture of an ARA-containing edible fat or oil and a marigold pigment raw material.

Table 4 shows the amount (g) of the oral composition compounded, the proportions (% by weight) of the components in the oral composition, and the weight ratios of the components. Table 5 shows the compositions of the glycerol fatty acid ester (C) and the (poly)glycerol fatty acid ester (C′), the number of unsaturated bonds, and the separation evaluation of the oral composition.

TABLE 4

| | | | Comparative |
|---|---|---|---|
| | | Examples | Examples |
| | | 16 to 21 | 11 to 20 |
| Amount | Arachidonic acid-containing edible fat or oil | 6.0 | 6.0 |
| (g) | DHA- and EPA-containing refined fish oil | 17.5 | 17.5 |
| | Marigold pigment raw material | 1.2 | 1.2 |
| | (C) or (C′) in Table 2 | 5.3 | 5.3 |
| | Total | 30 | 30 |
| Proportion of | Arachidonic acid (A) | 7.82 | 7.82 |
| component | Lutein and zeaxanthin (B) | 1.02 | 1.02 |
| (wt %) | Glycerol fatty acid ester (C) | 17.53 | — |
| | (Poly)glycerol fatty acid ester (C′) | — | 17.53 |
| Weight ratio of | (C)/(A) + (B) | 1.98 | — |
| components | (A)/(B) | 7.67 | 7.67 |

TABLE 5

| | | | | Number of | |
|---|---|---|---|---|---|
| | | | | unsaturated | Separation |
| | | Main componnet | Category | bonds | evaluation |
| | Glycerol fatty | | | | |
| | acid ester (C) | | | | |
| Example 16 | (C1) | Hydrogenated rapeseed oil fatty | Fatty acid mono/diglyceride | 0 | ∘∘ |
| | | acid mono/diglyceride | | | |
| Example 17 | (C2) | Self-emulsifying stearic acid | Fatty acid mono/diglyceride | 0 | ∘∘ |
| | | mono/diglyceride | | | |
| Example 18 | (C3) | Lauric acid monoglyceride | Fatty acid monoglyceride | 0 | ∘∘ |
| Example 19 | (C4) | Hydrogenated palm oil | Fatty acid monoglyceride | 0 | ∘∘ |
| | | monoglyceride | | | |
| Example 20 | (C5) | Lauric acid monoglyceride | Fatty acid monoglyceride | 0 | ∘∘ |
| Example 21 | (C6) | Stearic acid monoglyceride | Fatty acid monoglyceride | 0 | ∘∘ |
| | | Palmitic acid monoglyceride | | | |
| | (Poly)glycerol fatty | | | | |
| | acid ester (C′) | | | | |
| Comparative Example 11 | (C′1) | Diglycerol mono/distearate | Polyglycerol fatty acid ester | 0 | Δ |
| Comparative Example 12 | (C′2) | Decaglycerol decastearate | Polyglycerol fatty acid ester | 0 | x |
| Comparative Example 13 | (C′3) | Oleic acid monoglyceride | Fatty acid monoglyceride | 1 | x |
| Comparative Example 14 | (C′4) | Diglycerol mono/dioleate | Polyglycerol fatty acid ester | 1 | x |
| Comparative Example 15 | (C′5) | Oleic acid mono/diglyceride | Fatty acid mono/diglyceride | 1 | x |
| Comparative Example 16 | (C′6) | Caprylic acid monoglyceride | Fatty acid monoglyceride | 0 | x |
| Comparative Example 17 | (C′7) | Decaglycerol decaoleate | Polyglycerol fatty acid ester | 1 | x |
| Comparative Example 18 | (C′8) | Decaglycerol pentaoleate | Polyglycerol fatty acid ester | 1 | x |
| Comparative Example 19 | (C′9) | Diglycerol monolaurate | Polyglycerol fatty acid ester | 0 | x |
| Comparative Example 20 | (C′10) | Glycerol citrate monooleate | Organic acid monoglyceride | 1 | x |

As shown in Table 5, the oral compositions of Examples 16 to 21 did not separate. The oral compositions of Comparative Examples 11 to 20 completely separated or partially separated as with the oral compositions of Comparative Examples 1 to 10.

The glycerol fatty acid esters (C1) to (C6) used in Examples 16 to 21 were each a monoglyceride and/or a diglyceride containing a saturated fatty acid as a constituent fatty acid.

The results therefore have demonstrated that even the oral composition containing arachidonic acid, DHA, EPA, and lutein and/or zeaxanthin was less prone to separation owing to the presence of a monoglyceride and/or a diglyceride containing a C12-C24 saturated fatty acid as a constituent fatty acid.

The results also have confirmed that the oral compositions of the comparative examples tend to have low viscosity, while the oral compositions of the examples have moderate viscosity.

The following describes a production example of a soft capsule.

### Preparation of Soft Capsule

### Formulation Example 1

Water was added to 100 parts by weight of gelatin and 35 parts by weight of food additive grade glycerol. The gelatin is dissolved at 50 to 60° C., preparing a gelatin solution having a viscosity of 2000 cp. Next, raw materials are stirred and mixed with the following proportions: 30% by weight of an ARA-containing edible fat or oil derived from a microorganism (containing 40% arachidonic acid); 48% by weight of safflower oil; 4% by weight of a raw material containing 20% lutein derived from marigold; and 18% by weight of a glycerol fatty acid ester (containing 60% by weight of stearic acid monoglyceride as a main component). Thereby, an oral composition is prepared. Using the gelatin solution and the oral composition, capsule molding and drying are performed by a common method, producing soft capsules containing 200 mg of the oral composition per capsule as a filling.

In Formulation Example 1, the oral composition prepared in any of Examples 1 to 21 may be used to produce soft capsules, for example.

1. An oral composition comprising:
a component (A): an arachidonic acid-class compound;
a component (B): at least one compound selected from the group consisting of lutein, zeaxanthin, and fatty acid esters thereof; and
a component (C): a glycerol fatty acid ester (s),
wherein the glycerol fatty acid ester (s) is a monoglyceride and/or a diglyceride containing a C12-C24 saturated fatty acid as a constituent fatty acid.

2. The oral composition according to claim 1,
wherein the oral composition has a weight ratio of an amount of the component (C) to a sum of amounts of the component (A) (in terms of arachidonic acid) and the component (B) (in terms of free form) ((C)/((A)+(B))) of 0.40 to 20.

3. The oral composition according to claim 1,
wherein the arachidonic acid-class compound is at least one compound selected from the group consisting of arachidonic acid, a salt thereof, and a compound containing arachidonic acid as a constituent fatty acid.

4. The oral composition according to claim 1,
wherein the component (C) is contained in an amount of 4 to 25% by weight.

5. The oral composition according to claim 1,
wherein the oral composition is to be charged into a soft capsule.

6. A soft capsule comprising:
a filling containing the oral composition according to claim 1; and
a coating.

7. A method for producing an oral composition, the method comprising:
mixing a component (A): an arachidonic acid-class compound; a component (B): at least one compound selected from the group consisting of lutein, zeaxanthin, and fatty acid esters thereof; and a component (C): a glycerol fatty acid ester (s),
wherein the glycerol fatty acid ester (s) is a monoglyceride and/or a diglyceride containing a C12-C24 saturated fatty acid as a constituent fatty acid.
